(19) United States
(12) Patent Application Publication
(10) Pub. No.: US 2011/0207036 A1
(43) Pub. Date: Aug. 25, 2011

(54) Electrophoretic Particle Salt For Electrophoretic Display And Method Of Making

(76) Inventors: Yoocharn Jeon, Palo Alto, CA (US); Zhang-Lin Zhou, Palo Alto, CA (US)

(21) Appl. No.: 13/126,760

(22) PCT Filed: Oct. 30, 2008
(86) PCT No.: PCT/US2008/081710

§ 371 Date: Apr. 28, 2011

(51) Int. Cl. G03G 17/00; C07C 211/01; C07F 9/54; C07F 9/70; C07C 209/68; C07C 391/00; C07D 213/20; C07D 233/58; C07C 395/00

(52) U.S. Cl. 430/32; 564/291; 568/9; 556/70; 564/296; 562/899; 546/347; 548/335.1; 564/8; 556/7; 548/110; 546/13; 556/24

(57) ABSTRACT

An electrophoretic particle salt that includes a cationic electrophoretic particle and an anionic group ionically associated with the cationic electrophoretic particle is employed in an electrophoretic display. A spacer group chemically bonds a cationic moiety to a surface of the electrophoretic particle. A method of making the electrophoretic particle salt includes particle surface modification, nucleophilic substitution to create an interim salt and anion exchange. The electrophoretic particle salt has an ionization constant that favors dissociation into a positively charged electrophoretic particle and the anionic group in a nonpolar medium. The electrophoretic display includes a pair of electrodes and a dispersion of the electrophoretic particle salt in a nonpolar medium in a gap between the pair of electrodes.

### CROSS-REFERENCE TO RELATED APPLICATIONS

N/A

### STATEMENT REGARDING FEDERALLY SPONSORED RESEARCH OR DEVELOPMENT

N/A

### BACKGROUND

1. Technical Field

The invention relates to electrophoretic displays. In particular, the invention relates to a cationic electrophoretic particle in association with an anion in a salt.

2. Description of Related Art

Electrophoretic display systems generally rely on electrophoretic movement of one or more charged particles (e.g., charged pigment particles) in a carrier medium or ‘suspension’ to display information. In some instances, the charged particles are accompanied by counter ions created in the suspension when the particles are charged. Information is displayed by one or both of movement of the charged particles relative to the suspension (e.g., colored particles moving in a contrasting colored suspension) and differential movement of differently colored particles relative to one another. In general, particles used in electrophoretic displays may be either positively charged particles or negatively charged particles.

To impart a charge (either positive or negative) to the particles in suspension, a charge control agent(s) is typically added to the suspension. The charge control agent interacts with the particle to establish the charge on the particle. For example, a Bronsted base group may be included on the surface of the particle to produce a positively charged particle. The Bronsted base group will accept a positively charged hydrogen ion (i.e., a proton) from a proton donor species to create a positive charge on the particle. The charge control agent acts as the proton donor species in such systems.

Typically, an amount of charge control agent that must be added to the suspension necessary to charge the extant particles exceeds an equilibrium amount because not all of the charge control agent successfully interacts with (e.g., provides donor protons to) the particles to charge them. As such, excess charge control agent is usually added to the suspension to insure all of the particles are successfully charged. Unfortunately, adding excess charge control agent generally leads to the presence of excess charge in the suspension that is not associated with the charged particles. This excess charge may interfere with the operation of the electrophoretic display through effects such as, but not limited to, charge accumulation on the electrodes and electric field screening.

One example of a Bronsted base is an amine group on the surface of the particle. In suspension, the Bronsted base at the surface of the particle typically will accept a proton from a charge control agent (e.g., a positively charged ammonium compound). However, even with electrophoretic particles that have a Bronsted base group, there must be an excess amount of the charge control agent that acts as the proton donor species in suspension. The excess proton donor species facilitates the Bronsted base group on the particle to accept a proton, since not all protons released from the proton donor species will actually form a bond with the Bronsted base group on the particle.

As described above, the excess proton donor species tends to accumulate on the oppositely charged electrodes. The accumulation of charge on the electrodes interferes with electrophoretic display operation through electric field screening. As a consequence, the performance of the electrophoretic display degrades over time. Hence, a positively charged electrophoretic particle that does not need a donor species (i.e., charge control agent) to positively charge the electrophoretic particle in electrophoretic display applications would satisfy a long felt need.

### BRIEF SUMMARY

In an embodiment of the present invention, an electrophoretic particle salt is provided. The electrophoretic particle salt comprises an electrophoretic particle having a cationic moiety and a spacer group that chemically bonds the cationic moiety to a surface of the electrophoretic particle. The spacer group comprises a saturated hydrocarbon. The electrophoretic particle salt further comprises an anionic group ionically associated with the cationic moiety of the electrophoretic particle. The electrophoretic particle salt has an ionization constant that favors dissociation into a positively charged electrophoretic particle and the anionic group in a nonpolar medium.

In another embodiment of the present invention, an electrophoretic display is provided. The electrophoretic display comprises a pair of electrodes separated by a gap and the electrophoretic particle salt dispersed in a nonpolar medium in the gap between the pair of electrodes. The electrophoretic particle salt is ionically dissociated in the nonpolar medium, such that a negative ion is released and a positive charge is retained on the electrophoretic particle. A total charge generated by the electrophoretic particle salt in the nonpolar medium is compatible for electrophoretic display operation, such that inclusion of a charge control agent is avoided and one or both of electric field screening and excess charge accumulation during the electrophoretic display operation is reduced.

In another embodiment of the present invention, a method of making the electrophoretic particle salt is provided. The method of making comprises modifying a surface of an electrophoretic particle to chemically bond a spacer group and a moiety to the surface. The method of making further comprises creating an interim salt with the modified electrophoretic particle using nucleophilic substitution. The moiety on the modified electrophoretic particle is one of a nucleophile and a leaving group. The method of making further comprising exchanging a negative species from the interim salt with an anionic group to form the electrophoretic particle salt.

Certain embodiments of the present invention have other features that are one of in addition to and in lieu of the features described hereinabove. These and other features of the invention are detailed below with reference to the following drawings.

### BRIEF DESCRIPTION OF THE DRAWINGS

The various features of the embodiments of the present invention may be more readily understood with reference to the following detailed description taken in conjunction with the accompanying drawings, where like reference numerals designate like structural elements, and in which:

FIG. 1 illustrates a side view of an electrophoretic display, according to an embodiment of the present invention.

FIG. 2 illustrates a flow chart of a method of making an electrophoretic particle salt, according to an embodiment of the present invention.

FIG. 3A illustrates a flow chart of creating an interim salt of the method of FIG. 2, according to an embodiment of the present invention.

FIG. 3B illustrates a flow chart of creating an interim salt of the method of FIG. 2, according to another embodiment of the present invention.

### DETAILED DESCRIPTION

Embodiments of the present invention employ an electrophoretic particle salt in a dispersion medium that is used in electrophoretic displays. The electrophoretic particle salt ionically dissociates in a nonpolar medium into a positively charged electrophoretic particle and a negatively charged co-ion. In effect, the electrophoretic particle salt is self-charged or is self-charging in that the electrophoretic particle salt releases the negative co-ion and retains a positive charge on the electrophoretic particle for display operation. In other words, the electrophoretic particle salt provides essentially equivalent amounts of both positive charge species and negative charge co-ion species when dispersed in a nonpolar medium. As such, there may be essentially no excess charge of either species in the embodiments of the present invention.

A total charge created by the electrophoretic particle salt is compatible with electrophoretic display operation according to the embodiments of the present invention. By ‘compatible’ it is meant that the electrophoretic particle salt provides a sufficient amount of both positively charged species and negatively charged species to adequately operate an electrophoretic display. As such, inclusion of a charge control agent is avoided and unnecessary. Moreover, ‘compatible’ means that the electrophoretic particle salt reduces, and in some embodiments minimizes, one or both of electric field screening and excess charge accumulation on electrodes during the electrophoretic display operation.

The electrophoretic particle salt is made using a combination of particle surface modification, nucleophilic substitution and anion exchange reactions. A spacer group is chemically bonded to a surface of the electrophoretic particle. The spacer group is further chemically bonded to a cationic moiety. The cationic electrophoretic particle is ionically associated with an anionic compound or group to form the salt. The ionization constant for the electrophoretic particle salt is conducive to dissociating in a nonpolar medium. Upon dissociation, the electrophoretic particle salt releases the anionic group (i.e., the co-ion species) and retains a positive charge on the electrophoretic particle. The anion group and the cationic electrophoretic particle in the nonpolar medium are available to move in response to an electric field between oppositely charged electrodes of an electrophoretic display.

According to various embodiment of the present invention, the electrophoretic particle includes organic and inorganic colored pigments and organic colored polymers that can undergo a surface modification to chemically bond to a cationic moiety by way of a spacer group. All possible colors that fall within one or both of an RGB color model (red-green-blue) and a CMYK color model (cyan-magenta-yellow-black) are within the scope of the pigments and polymers useful herein. The inorganic pigments used for electrophoretic particles include, but are not limited to, titanium oxide, carbon black, molybdenum red, titanium cobalt green, Prussian blue, and cadmium yellow. Organic pigments used for electrophoretic particles include, but are not limited to, phthalocyanine dyes and azo pigments. Moreover, some organic colored polymers (plastics) used for electrophoretic particles include, but are not limited to, methylacrylates, methylacrylic acids, various alkenoic acids and copolymers of various acids and acrylates. In some embodiments, the electrophoretic particle has a particle size ranging from 50 nanometers and 1 micron.

In various embodiments of the present invention, the spacer group is a moiety that makes a chemical bond to a surface of the electrophoretic particle as well as to a cationic moiety. For example, the spacer group has opposite ends available for bonding, wherein one end of the spacer group is chemically bonded to the electrophoretic particle surface while an opposite end is chemically bonded to the cationic moiety. In some embodiments, the chemical bond is a covalent bond to one or both of the particle and the cationic moiety. In other embodiments, the chemical bond is at least strong enough to withstand breaking in both a nonpolar medium and under the influence of an electric field (e.g., as in an electrophoretic display). In some embodiments, the spacer group is a pure hydrocarbon (i.e., comprises only carbon and hydrogen). In some embodiments, the hydrocarbon spacer group is a saturated hydrocarbon (i.e., an alkane or an alkyl group). The saturated hydrocarbon has a chemical structure —(CH₂)ₙ— where n ranges from 1 to 25, in some embodiments. Moreover, the saturated hydrocarbon may be one of straight chain, branched chain and a ring structure. In other embodiments, the spacer group is a hydrocarbon including, but not limited to, an alkyl group, an alkenyl group, an alkynyl group, a cycloalkyl group and aryl group.

The cationic moiety, according to the various embodiments herein, comprises one of nitrogen, phosphorus, arsenic, selenium, and tellurium. In some embodiments, the nitrogen-based cationic moiety includes, but is not limited to, a quaternary ammonium cation (i.e., —N⁺R₁R₂R₃ or (R)₃-substituted quaternary ammonium cation), an R-substituted pyridinium cation, and an R-substituted imidazolium cation. In some embodiments, the phosphorus-based cationic moiety includes, but is not limited to, a quaternary phosphonium cation (i.e., —P⁺R₁R₂R₃ or (R)₃-substituted phosphonium cation).

In some embodiments, the arsenic-based cationic moiety includes, but is not limited to, —As⁺R₁R₂R₃. In some embodiments, the selenium-based cation includes, but is not limited to, —Se⁺R₁R₂. In some embodiments, the tellurium-based cation includes, but is not limited to, —Te⁺R₁R₂. Each ‘R’ (i.e., R, R₁, R₂, R₃) substitution of the cationic moiety is independently selected from hydrogen and an organic substituent. The organic substituent is either a branched group or an unbranched group including, but not limited to, alkyl, alkenyl, alkynyl, cyclo, aryl, and hetero versions of any of these groups that include one or more of sulfur (S), nitrogen (N) and oxygen (O), for example.

In some embodiments, the unbranched alkyl R group includes, but is not limited to, methyl, ethyl, propyl, butyl, n-octyl, n-decyl, n-dodecyl, and n-tetradecyl. The branched alkyl R group includes, but is not limited to, isopropyl and iso-butyl, in some embodiments. In some embodiments, the number of carbons in the organic substituent R group may range from 1 to 25.

According to the various embodiments, the anionic group (i.e., the ‘co-ion’) that is ionically associated with the cationic moiety of the electrophoretic particle of the salt will readily dissociate from the cationic electrophoretic particle in a nonpolar medium. In other words, the electrophoretic particle salt has an ionization constant that favors dissociation in the nonpolar medium. In some embodiments, the anionic group includes, but is not limited to, a halogen ion, a hydroxide ion, a carboxylic acid ion, a phosphoric acid ion, a sulfuric acid ion, a hexafluorophosphoric acid ion, and a tetraphenyl boronic ion.

The nonpolar medium for the various embodiments of the present invention comprises one of a hydrocarbon, an aliphatic hydrocarbon, and an isomerized aliphatic hydrocarbon that includes, but is not limited to, dodecane, cyclohexane, Isopar G, Isopar H, Isopar L, Isopar M and Isopar V. Isopar is a brand name for a range of isoparaffinic fluids offered by ExxonMobil Chemical. ISOPAR® is a registered trademark of Exxon Mobil Corporation, Irving, Tex.

For simplicity herein, no distinction is made between the term ‘species’ as referring to a single item (e.g., a single particle, counter-ion, etc.) and a plurality of such items unless such a distinction is necessary for proper understanding. Further, as used herein, the article ‘a’ is intended to have its ordinary meaning in the patent arts, namely ‘one or more’. For example, ‘a particle’ generally means one or more particles and as such, ‘the particle’ means ‘the particle(s)’ herein. Also, any reference herein to ‘top’, ‘bottom’, ‘upper’, ‘lower’, ‘up’, ‘down’, ‘left’ or ‘right’ is not intended to be a limitation herein. Moreover, examples herein are intended to be illustrative only and are presented for discussion purposes and not by way of limitation.

In some embodiments of the present invention, an electrophoretic particle salt is provided. The electrophoretic particle salt comprises an electrophoretic particle having a cationic moiety and a spacer group. The spacer group is chemically bonded to a surface of the electrophoretic particle. The cationic moiety is chemically bonded to the spacer group. The spacer group comprises a saturated hydrocarbon. The electrophoretic particle salt further comprises an anionic group ionically associated with the cationic moiety that is chemically bonded to the electrophoretic particle. The electrophoretic particle salt has an ionization constant that favors dissociation into a positively charged electrophoretic particle and a negatively charged co-ion (i.e., the anionic group) in a nonpolar medium. Any of the electrophoretic particles, the spacer groups, the cationic moieties and the anionic groups described above may be used for the various embodiments of the electrophoretic particle salt.

The electrophoretic particle salt is self-charged in a nonpolar medium, as defined above. In some embodiments, the electrophoretic particle salt further comprises a nonpolar medium that disperses the electrophoretic particle salt. Any of the nonpolar media described above may be used for the nonpolar dispersion medium, depending on the embodiment. A total charge generated by the electrophoretic particle salt in the nonpolar dispersion medium is compatible for electrophoretic display operation. In some embodiments, the total charge is made up of essentially equivalent amounts of positive charge and negative charge generated by the cationic electrophoretic particle and anionic group, respectively. In some of these embodiments, the total charge in the electrophoretic system is provided exclusively by the aforementioned cationic electrophoretic particle and anionic group of the electrophoretic particle salt embodiments of the present invention. The compatibility of the electrophoretic particle salt with electrophoretic display operation means that use of a charge control agent is unnecessary, and therefore avoided, and that one or both of field screening and excess charge accumulation during electrophoretic display operation is reduced. In some embodiments, one or both of field screening and excess charge accumulation is minimized during electrophoretic display operation.

In other embodiments of the present invention, an electrophoretic dispersion is provided. The electrophoretic dispersion comprises a salt of an electrophoretic particle and a nonpolar medium that disperses the salt. The salt comprises an electrophoretic particle having a cationic moiety and a spacer group that chemically bonds the cationic moiety to a surface of the electrophoretic particle. The salt further comprises an anionic group ionically associated with the cationic moiety that is bonded to the electrophoretic particle. The spacer group is a saturated hydrocarbon. Any of the respective materials provided above are useful for the salt of an electrophoretic particle. In some embodiments, the salt of an electrophoretic particle is the same as any of the electrophoretic particle salt embodiments described above. The dispersed salt is ionically dissociated in the nonpolar medium into a positively charged electrophoretic particle and the anionic group. Any of the nonpolar media described above may be used for the electrophoretic dispersion embodiments.

The electrophoretic dispersion is placed in a gap between a pair of electrodes of an electrophoretic display. Since the salt is self-charged in the nonpolar medium, the salt provides a sufficient amount of both positive charge species and negative charge species for operation of the electrophoretic display. The amount of respective charged species is compatible with electrophoretic display operation, such that inclusion of a charge control agent into the electrophoretic dispersion is circumvented.

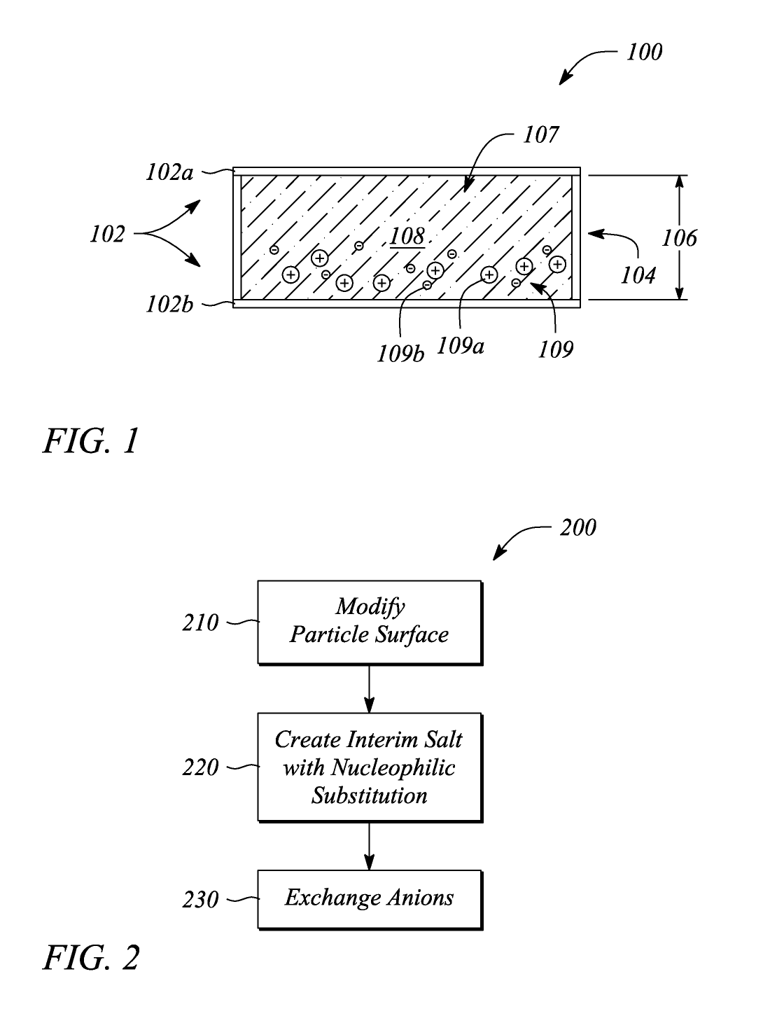

In other embodiments of the present invention, an electrophoretic display is provided. FIG. 1 illustrates a side view of the electrophoretic display 100, according to an embodiment of the present invention. The electrophoretic display 100 comprises a pair 102 of electrodes at opposite ends of a display housing 104. The electrodes 102a, 102b are separated by a gap 106 in the display housing 104. The electrophoretic display 100 further comprises an electrophoretic dispersion 108 in the gap 106 of the display housing 104 between the pair 102 of electrodes.

The electrophoretic dispersion 108 comprises a salt of an electrophoretic particle and a nonpolar medium 107 that disperses the electrophoretic particle salt 109. The electrophoretic particle salt 109 is ionically dissociated in the nonpolar medium 107 by releasing a negative ion 109b and retaining a positive charge on the electrophoretic particle 109a. A total charge generated by the electrophoretic particle salt 109 in the electrophoretic dispersion is compatible for electrophoretic display operation. In other words, a sufficient amount of both positive charge species 109a and negative charge species 109b is provided by the salt 109, such that a charge control agent need not be added to the electrophoretic dispersion 108 in order to operate the electrophoretic display 100. The sufficient amount of respective charge species 109a, 109b provided by the electrophoretic particle salt 109 avoids use of charge control agents and reduces one or both of field screening and excess charge accumulation on the electrodes of the electrophoretic display.

The electrophoretic particle salt 109 comprises an electrophoretic particle, a cationic moiety 109a and a spacer group that chemically bonds the cationic moiety 109a to a surface of the electrophoretic particle. The salt 109 further comprises an anionic group 109b ionically associated with the cationic moiety 109a attached to the surface of the electrophoretic particle. In some embodiments, the electrophoretic dispersion 108 is equivalent to any of the electrophoretic dispersion embodiments described above. In some embodiments, the electrophoretic particle salt 109 is the same as any of the embodiments described above for the electrophoretic particle salt.

In other embodiments of the present invention, a method of making an electrophoretic particle salt is provided. FIG. 2 illustrates a flow chart of the method 200 of making an electrophoretic particle salt according to an embodiment of the present invention. The method 200 of making comprises modifying 210 a surface of an electrophoretic particle with a moiety. Modifying 210 a surface comprises chemically bonding a spacer group to the electrophoretic particle surface. The spacer group has the moiety chemically bonded to the spacer group. In some embodiments, the spacer group is a saturated hydrocarbon that comprises the moiety at a terminus of the hydrocarbon spacer.

In some embodiments, the spacer group is chemically bonded to the electrophoretic particle surface using diazonium chemistry. For example, first, a diazonium salt of a spacer group ‘A’ is made. The spacer group A has the moiety ‘M’ attached, for example, at an end opposite to the diazonium group ‘N≡N⁺—’ (e.g., N≡N⁺-A-M). Second, in a reaction between the diazonium salt of the spacer group A and the electrophoretic particle ‘EP’, the spacer group A is bonded to the surface of the electrophoretic particle EP that in some embodiments, may include a release of nitrogen gas N₂ (i.e., ‘EP-A-M’ or ‘modified electrophoretic particle’ herein), as shown in equation (1), by way of example:

EP+⁺N≡N-A-M→EP-A-M+N₂ (1)

The moiety M remains attached to the spacer group during the surface modification of the electrophoretic particle EP and is available for subsequent reaction, as described further below.

The method 200 of making further comprises creating 220 an interim salt of the modified electrophoretic particle using nucleophilic substitution. Creating 220 an interim salt comprises a forming a salt between a nucleophile and a leaving group, wherein the moiety M on the modified electrophoretic particle is either the nucleophile or the leaving group, depending on the embodiment. The term ‘leaving group’ has its ordinary meaning in chemical practice for the purposes of the present invention. The terms ‘nucleophile’ and ‘nucleophilic group’ have their ordinary meaning in chemical practice for the purposes of the present invention also. The interim salt comprises a positively charged species on the surface of the modified electrophoretic particle and a negatively charged species, wherein the negatively charged species is a negatively charged leaving group.

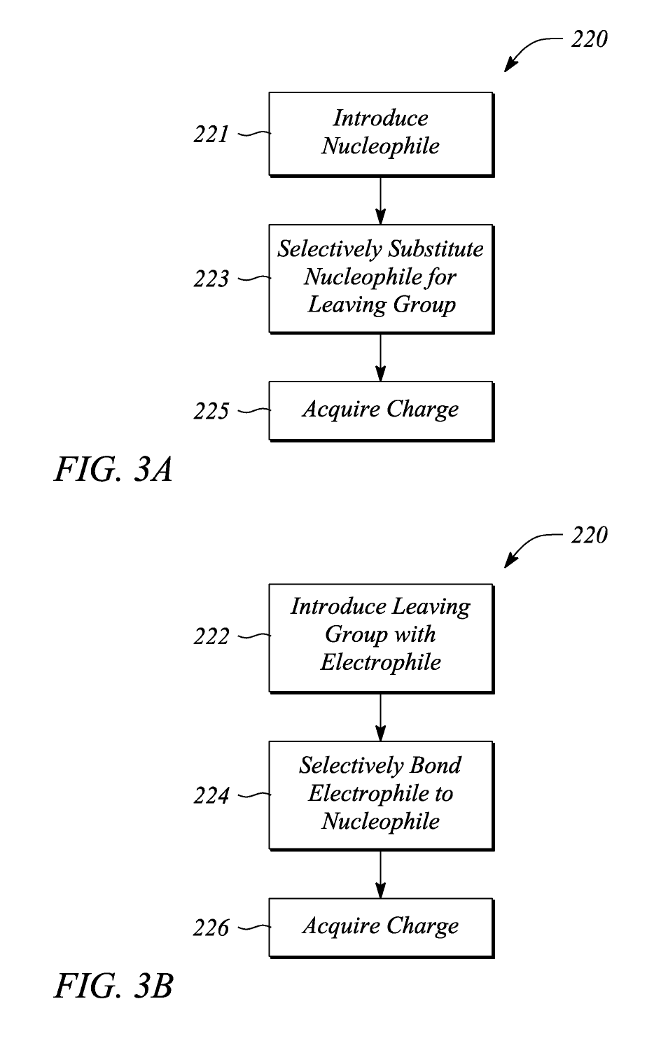

In some embodiments, the moiety M attached to the modified electrophoretic particle by way of the spacer group A is the leaving group (i.e., M=LG). FIG. 3A illustrates a flow chart of creating 220 an interim salt using nucleophilic substitution of the method 200 in FIG. 2, according to an embodiment of the present invention. In the embodiment of FIG. 3A, nucleophilic substitution comprises introducing 221 a nucleophile Y to the modified electrophoretic particle, such that the nucleophile Y substitutes 223 for the leaving group LG on the modified electrophoretic particle and selectively bonds with the spacer group A (an electrophile) and during creating 220 an interim salt. The released leaving group acquires 225 a negatively charge LG⁻ and is the negatively charged species of the created 220 interim salt. The nucleophile acquires 225 a positive charge Y⁺ and is the positively charged species on the modified electrophoretic particle of the created 220 interim salt, as shown in equation (2):

EP-A-LG+Y→EP-A-Y⁺LG⁻ (2)

In other embodiments, the moiety M attached to the modified electrophoretic particle is the nucleophile (i.e., M=Y). FIG. 3B illustrates a flow chart of creating 220 an interim salt using nucleophilic substitution of the method 200 in FIG. 2, according to another embodiment of the present invention. In the embodiment of FIG. 3B, nucleophilic substitution comprises introducing 222 a leaving group LG that comprises an electrophilic species R₀ (i.e., LG-R₀) to the modified electrophoretic particle, such that the nucleophile Y on the modified electrophoretic particle selectively bonds 224 with the electrophilic species R₀ from the leaving group LG during creating 220 an interim salt. The nucleophile acquires 226 a positive charge Y⁺—R₀ and is the positively charged species on the modified electrophoretic particle of the created 220 interim salt. The remaining leaving group acquires 226 a negative charge LG⁻ and is the negatively charged species of the created 220 interim salt, as shown in equation (3):

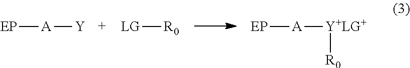

In some embodiments, the leaving group LG comprises one of chloride, bromide, iodide, p-toluenesulfonyl, and trifluoromethanesulfonyl. In some embodiments, the electrophilic species R₀ comprises a hydrogen and an organic substituent, similar to the R substituent group of the cationic moiety described above. In some embodiments, the organic electrophilic species R₀ is independently one of an unbranched alkyl group and a branched alkyl group having from 1 to 25 carbons.

In some embodiments, the nucleophile Y comprises one of nitrogen, phosphorus, arsenic, selenium, and tellurium. For example, the nucleophile Y includes, but is not limited to, ammonia (NH₃), phosphine (PH₃), arsine (ArH₃), hydrogen selenide (SeH₂), hydrogen telluride (TeH₂), and organic R group substituted ones of nitrogen, phosphorus, arsenic, selenium, and tellurium. In some embodiments, the nucleophile Y may be a primary, secondary or tertiary amine, such that a quaternary ammonium cation is formed on the electrophoretic particle salt. In some embodiments, the nucleophile Y is a precursor of the cationic moiety described above. For example, the nucleophile Y includes, but is not limited to, pyridine and imidazole, such that a Pyridium cation or a imidazolium cation, respectively, is the positively charged species on the modified electrophoretic particle of the created 220 interim salt, depending on the embodiment.

The method 200 of making an electrophoretic particle salt further comprises exchanging 230 the negatively charged species (LG⁻) of the interim salt with an anionic group (X⁻) to form the electrophoretic particle salt. The electrophoretic particle salt made by the method 200 comprises the positively charged electrophoretic particle species and the anionic group X⁻ (i.e., ‘co-ion’) ionically associated with the positively charged electrophoretic particle species. According to the various embodiments herein, the anionic group X⁻ will readily exchange 230 with the negatively charged species LG⁻ on the interim salt and be ionically associated with the positively charged species of the electrophoretic particle in the electrophoretic particle salt made by the method 200, as shown in equations (4a) and (4b):

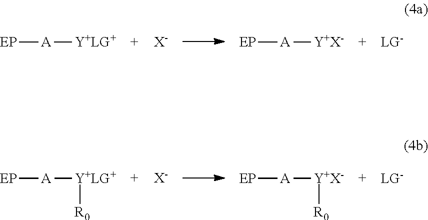

In some embodiments, the anionic group X⁻ that is exchanged 230 with the negatively charged species LG⁻ comprises an anion of one of a halogen, a hydroxide, a carboxylic acid, a phosphoric acid, a sulfuric acid, a hexafluorophosphoric acid, and a tetraphenyl boron. In some embodiments, the electrophoretic particle salt made by the method 200 of making is the same as any of the embodiments of the electrophoretic particle salt described above. The negatively charged leaving group LG⁻ is readily removed from the reaction mixture containing the electrophoretic particle salt after the anionic exchange 230 reaction. For example, the negatively charged leaving group LG⁻ is removed from the reaction mixture using ion exchange chromatography, wherein the negatively charged leaving group LG⁻ remains associated with an ion-exchange resin in a chromatography column and the electrophoretic particle salt moves through and exits the column.

Thus, there have been described embodiments of an electrophoretic particle salt having a cationic electrophoretic particle in ionic association with an anionic group at the particle surface; an electrophoretic display employing an electrophoretic dispersion of the salt; and a method of making the salt. It should be understood that the above-described embodiments are merely illustrative of some of the many specific embodiments that represent the principles of the present invention. Clearly, those skilled in the art can readily devise numerous other arrangements without departing from the scope of the present invention as defined by the following claims.

What is claimed is:

1. An electrophoretic particle salt comprising:
an electrophoretic particle having a cationic moiety and a spacer group that chemically bonds the cationic moiety to a surface of the electrophoretic particle, the spacer group comprising a saturated hydrocarbon; and
an anionic group ionically associated with the cationic moiety of the electrophoretic particle, the electrophoretic particle salt having an ionization constant that favors dissociation into a positively charged electrophoretic particle and the anionic group in a nonpolar medium.

2. The electrophoretic particle salt of claim 1, wherein the electrophoretic particle salt is self-charged, such that a charge control agent is unnecessary in an electrophoretic display comprising the electrophoretic particle salt in the nonpolar medium.

3. The electrophoretic particle salt of claim 1, wherein the electrophoretic particle comprises one or more of a colored pigment and a colored polymeric particle having a particle size ranging from 50 nanometers and 1 micron.

4. The electrophoretic particle salt of claim 1, wherein the spacer group has a chemical structure —(CH₂)ₙ—, where n ranges from 1 to 25, one end of the spacer group being chemically bonded to a surface of the electrophoretic particle and an opposite end of the spacer group being chemically bonded to the cationic moiety, and wherein the cationic moiety comprises one of nitrogen, phosphorus, arsenic, selenium, and tellurium.

5. The electrophoretic particle salt of claim 1, wherein the cationic moiety is selected from one of an (R)₃-substituted quaternary ammonium ion, an (R)₃-substituted phosphonium ion, an (R)₃-substituted arsinium ion, an (R)₂-substituted selenium-based ion, an (R)₂-substituted tellurium-based ion, an R-substituted pyridinium ion and an R-substituted imidazolium ion, each R being independently selected from hydrogen and an alkyl group that is either branched or unbranched.

6. The electrophoretic particle salt of claim 5, wherein the alkyl group is independently selected from methyl, ethyl, propyl, isopropyl, butyl, iso-butyl, n-octyl, n-decyl, n-dodecyl, and n-tetradecyl.

7. The electrophoretic particle salt of claim 1, wherein the anionic group comprises a negative ion of one of a halogen, a hydroxide, a carboxylic acid, a phosphoric acid, a sulfuric acid, a hexafluorophosphoric acid, and a tetraphenyl boron.

8. The electrophoretic particle salt of claim 1, further comprising the nonpolar medium that disperses the electrophoretic particle salt.

9. An electrophoretic display comprising:
a pair of electrodes separated by a gap in a display housing; and
an electrophoretic dispersion in the gap between the pair of electrodes, the electrophoretic dispersion comprising a salt of an electrophoretic particle and a nonpolar medium that disperses the electrophoretic particle salt, the electrophoretic particle salt being ionically dissociated in the nonpolar medium, such that a negative ion is released and a positive charge is retained on the electrophoretic particle,
wherein a total charge of the electrophoretic particle salt in the electrophoretic dispersion is compatible for electrophoretic display operation, such that one or both of field screening and excess charge accumulation during the electrophoretic display operation is reduced.

10. The electrophoretic display of claim 9, wherein the electrophoretic particle salt comprises:
an electrophoretic particle having a cationic moiety and a spacer group that chemically bonds the cationic moiety to a surface of the electrophoretic particle, the spacer group being a saturated hydrocarbon; and
an anionic group ionically associated with the cationic moiety on the electrophoretic particle, the electrophoretic particle salt being self-charged in the nonpolar medium, such that inclusion of a charge control agent into the nonpolar medium is circumvented.

11. The electrophoretic display of claim 10, wherein the cationic moiety on the electrophoretic particle surface comprises an R-substituted ion of one of nitrogen, phosphorus, arsenic, selenium, and tellurium, where each R is independently selected from hydrogen and an alkyl group that is either branched or unbranched,
wherein the spacer group has a chemical structure —(CH₂)ₙ—, where n ranges from 1 to 25, one end of the spacer group being chemically bonded to the surface of the electrophoretic particle, an opposite end of the spacer group being chemically bonded to the cationic moiety, and
wherein the anionic group comprises a negative ion of one of a halogen, a hydroxide, a carboxylic acid, a phosphoric acid, a sulfuric acid, a hexafluorophosphoric acid, and a tetraphenyl boron.

12. A method of making an electrophoretic particle salt comprising:
modifying a surface of an electrophoretic particle with a saturated hydrocarbon spacer and a moiety at a terminus of the hydrocarbon spacer;
creating an interim salt of the modified electrophoretic particle using nucleophilic substitution, the moiety on the modified electrophoretic particle being one of a nucleophile and a leaving group; and
exchanging a negative species from the interim salt with an anionic group to form the electrophoretic particle salt.

13. The method of making of claim 12, wherein the moiety attached to the modified electrophoretic particle is the leaving group, and wherein the nucleophile is substituted for the leaving group on the modified electrophoretic particle during creating an interim salt, the substituted nucleophile acquiring a positive charge of the created interim salt.

14. The method of making of claim 12, wherein the moiety attached to the modified electrophoretic particle is the nucleophile, the leaving group having an electrophilic species, and wherein the nucleophile on the modified electrophoretic particle selectively bonds to the electrophilic species from the leaving group during creating an interim salt, the nucleophile acquiring a positive charge of the created interim salt.

15. The method of making of claim 14, wherein the leaving group comprises one of chloride, bromide, iodide, p-toluenesulfonyl, and trifluoromethanesulfonyl, the electrophilic species comprising a hydrogen, an alkyl group and a branched alkyl group.
